(19) United States
(12) Patent Application Publication
(10) Pub. No.: US 2021/0069237 A1
(43) Pub. Date: Mar. 11, 2021

(54) PHARMACEUTICAL COMPOSITIONS COMPRISING HEPARINOIDS AND METHODS FOR PREPARING THEREOF

(71) Applicant: Harrow IP, LLC, Nashville, TN (US)

(72) Inventor: Dennis Elias Saadeh, Brentwood, TN (US)

(21) Appl. No.: 17/009,318

(22) Filed: Sep. 1, 2020

Related U.S. Application Data
(60) Provisional application No. 62/897,519, filed on Sep. 9, 2019.

(51) Int. Cl. A61K 31/737; A61K 47/14; A61K 47/32; A61K 47/38; A61K 9/48; A61K 47/44; A61K 47/26; A61K 47/10; A61K 9/00

(52) U.S. Cl. CPC A61K 31/737; A61K 47/14; A61K 47/32; A61K 47/38; A61K 9/0053; A61K 47/44; A61K 47/26; A61K 47/10; A61K 9/4866

(57) ABSTRACT

Pharmaceutical compositions are described herein, among others, the compositions comprising anhydrous suspensions of a therapeutically effective quantity of a heparinoid, or a derivative or analog thereof, and to methods of preparing and using such compositions. The use of various heparinoids for these purposes is disclosed, such as pentosan, heparin, hyaluronic acid, chondroitin, and pharmaceutically acceptable salts thereof. Methods for fabricating the compositions and using them are also described.

### CROSS-REFERENCE TO RELATED APPLICATIONS

This application claims priority under 35 U.S.C. § 119(e) to U.S. Provisional Application No. 62/897,519, filed on Sep. 9, 2019, the entire content of which is hereby incorporated by reference.

### FIELD OF THE INVENTION

The present invention relates generally to the field of pharmaceuticals, and more specifically, to pharmaceutical compositions that include as an active component a therapeutically effective quantity of a heparinoid, or a derivative or analog thereof, and to methods of preparing and using such compositions.

### BACKGROUND

Interstitial cystitis is a serious disorder of the lower urinary tract that causes urinary urgency as well as pelvic pain. The condition is quite common and the number of patients who are suffering from it is estimated to be more than 1 million.

Traditionally, interstitial cystitis has been treated with dimethylsulfoxide for more than 40 years. Treatment using dimethylsulfoxide includes weekly intravesical instillations for 6 to 8 weeks followed by biweekly instillations for a maintenance period of 3-12 months.

However, dimethylsulfoxide therapy is limited in that it provides beneficial results for not more than half of the patients treated and the treatment takes a long time to reduce symptoms. Furthermore, this therapy may also be painful for many patients, with local anesthetics being ineffective in reducing the pain due to their lack of absorption into the bladder wall. Stronger, narcotic-type anesthetics have also proven to be only minimally effective. In addition, for the dimethylsulfoxide treatments to be more effective patients are also required to make serious changes in their lifestyle, such as avoiding potassium-rich foods, e.g., citrus fruits, tomatoes, chocolate, and coffee.

Accordingly, there exists a need for improved treatments that would at a minimum benefit a larger portion of the patient population, provide better relief with less pain, and avoid the need for extensive alterations in diet. This patent specification discloses such pharmaceutical compositions of heparinoids, and methods of fabricating and administering the same and addresses the drawbacks of the existing formulations.

### SUMMARY

According to exemplary embodiments of the invention, a pharmaceutical composition formulated as an anhydrous suspension is provided. In various embodiments, the composition includes a dispersed phase comprising a therapeutically effective quantity of a pharmaceutically acceptable heparinoid, and an anhydrous dispersion medium (which may optionally include at least one pharmaceutically acceptable surfactant, solubilizing agent, and/or suspending agent), in which the dispersed phase is dispersed.

In various embodiments, the heparinoid may be any of pentosan, heparin, hyaluronic acid, chondroitin, pharmaceutically acceptable salts thereof, and any combination thereof. In various embodiments of the invention, the anhydrous dispersion medium includes at least one of a vegetable oil (e.g., castor oil, soybean oil, coconut oil, avocado oil, olive oil, almond oil) or a medium chain triglyceride.

In various embodiments of the invention, the acceptable surfactant, solubilizing agent, and/or suspending agent, if used, may be any of non-ionic polyoxyethylene-polyoxypropylene block copolymers, a water-soluble derivative of cellulose, optionally partially cross-linked polyacrylates, polyoxyethylene sorbitan monolaurates, polyoxyethylene sorbitan monopalmitates, polyoxyethylene sorbitan monostearates, polyoxyethylene sorbitan monooleates, glyceryl distearate, triglycerol monooleate, and combinations thereof.

According to various other embodiments of the present application, the pharmaceutical compositions described herein are formulated as aqueous solutions comprising at least one heparinoid dissolved in water and may include further optional excipients and/or additives.

According to various further embodiments of the invention, the pharmaceutical compositions described herein may be administered orally, or alternatively, via any of various injections, such as intravenous, intramuscular, or intraarticular injections, to a mammalian subject in need of such treatment for treating interstitial cystitis or osteoarthritis.

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20, as well as to the fractional numbers in between integers, e.g., 1.5 or 2.5, and the like.

The term “pharmaceutical composition” is defined as a chemical or a biological compound or substance, or a mixture or combination of two or more such compounds or substances, intended for use in the medical diagnosis, cure, treatment, or prevention of disease or pathology.

The term “suspension” is defined for the purposes of the present application as a solid-in-liquid dispersion system having a first phase and a second phase, and optionally one or more additional phases. The above mentioned first phase consists of a multitude of solid particles and is designated and defined as the dispersed phase, and the above mentioned second phase is a liquid and is designated and defined as the dispersion medium, or, interchangeably and synonymously, the continuous phase. Thus, in other words, a suspension may be described as a mixture in which a multitude of solid particles are suspended throughout the bulk of a liquid phase. Embodiments with more than two phases may have a first solid phase and at least one additional solid phase (e.g., a second solid phase, a third solid phase, etc.), wherein each solid phase consists of a multitude of solid particles but the composition of the solid particles differs between the phases. For instance, a first solid phase may have a first active ingredient and a second solid phase may have a second active ingredient, wherein at least one of the active ingredients is a heparinoid. As another example, both a first and second solid phase may have the same active ingredient (i.e., a heparinoid) but the particles may be formulated to provide different rates of drug release. The term “stable” in reference to a suspension of the present application means that one or more characteristics of the heparinoid, the dispersed phase, and/or the dispersion medium does not significantly change over a period of a prolonged period of time (e.g., at least 30 days). Minimally, in a stable suspension the concentration of heparinoid, as measured in a sample obtained from the suspension, does not deviate by more than ±15%, or preferably ±10%, after storage at room temperature for at least 30, 60, 90, or 180 days. In addition, the dispersed phase may be evenly distributed within the continuous phase throughout the entire volume of the suspension.

The term “polydispersity index” or “PDI” refers to the degree of homogeneousness of a polymer sample and is calculated as the ratio between the weight-average molecular weight and the number-average molecular weight of a particular sample of the polymer. For monodisperse polymers the PDI is 1 by definition. As guidance only, the best controlled synthetic polymers typically have the PDI of 1.02-1.10.

As used herein, the term “heparinoid” refers to any molecule comprising a glycosaminoglycan (GAG) moiety, which is a moiety that comprises linear polysaccharides consisting of a repeating chain of disaccharide units. For illustrative purposes, one representative example of a specific compound that contains a GAG moiety is shown below (hyaluronan):

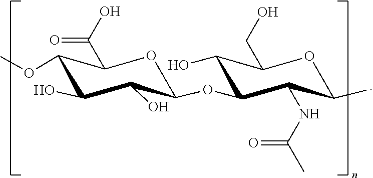

For purposes of the present disclosure, glycosaminoglycans are not limited to any one GAG or any specific source of GAG; therefore, GAG molecules include but are not limited to low molecular weight (i.e., in the range of about 2,000-8,000 Daltons), naturally derived, biotechnologically prepared, chemically modified, synthetic, etc., glycosaminoglycans, and the like. The present invention is not limited to any one specific heparinoid molecule or to any one specific source thereof.

As used herein, “heparin” refers to a heterogeneous group of straight-chained anionic glycosaminoglycans, as described above, with a molecular weight ranging between about 2,000 to 40,000 Daltons. Heparin, being (2R,3R,4R,5S,6S)-6-[(2R,3R,4R,5R,6S)-6-[(2S,3 S,4S,5R,6R)-6-[(2S,3 S,4R,5R,6R)-5-acetamido-4,6-dihydroxy-2-(sulfooxymethyl)oxan-3-yl]oxy-2-carboxy-4-hydroxy-5-sulfooxy oxan-3-yl]oxy-2-(hydroxymethyl)-5-(sulfoamino)-4-sulfooxyoxan-3-yl]oxy-3,4-dihydroxy-5-sulfooxyoxane-2-carboxylic acid (according to the IUPAC), is shown below in the form of a polysulfate salt:

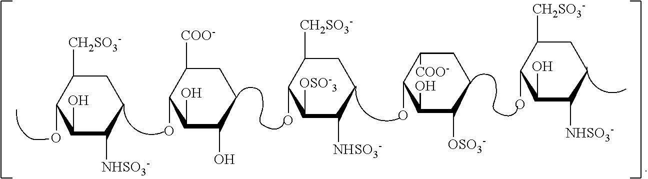

As used herein, “pentosan” is a derivative of a polysaccharide xylan, which is [(2R,3R,4S,5R)-2-hydroxy-5-[(2S,3R,4S,5R)-5-hydroxy-3,4-disulfooxyoxan-2-yl]oxy-3-sulfooxyoxan-4-yl] hydrogen sulfate (IUPAC), pentosan having a general structure shown below in form of a polysulfate salt:

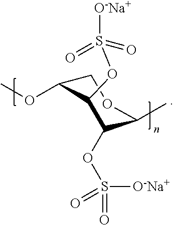

The term “medium-chain triglycerides” refers to triglycerides in which at least two of the three fatty acids are medium-chain fatty acids. Medium chain fatty acids have a linear, carbon chain backbone of 6-12 carbon atoms, which is either saturated or unsaturated. Non-limiting examples of medium-chain fatty acids include caproic (IUPAC, hexanoic), enanthic (IUPAC, heptanoic), caprylic (IUPAC, octanoic), pelargonic (IUPAC, nonanoic), capric (IUPAC, decanoic), undecylic (IUPAC, undecanoic), or lauric (IUPAC, dodecanoic) acids.

The term “carrier” refers to a substance that serves as a vehicle for improving the efficiency of delivery and the effectiveness of a pharmaceutical composition.

The term “solubilizing agent” for the purposes of the instant application refers broadly to chemical compounds that improve the process of incorporating the solubilizate (i.e., active components described herein) into micelles; in other words, the presence of a solubilizing agent makes the process of solubilization faster, easier, and/or more complete compared with compositions without it.

The term “suspending agent” is used herein interchangeably with the term “emulsifier” for the purposes of the instant application and refers broadly to chemical compounds that help active pharmaceutical ingredients stay suspended in the formulation and prevent and/or reduce the phase separation of two-phase dispersion systems described herein.

The term “interstitial cystitis” refers to a chronic progressive disorder of the lower urinary tract that causes urinary urgency and frequency and often also causes pelvic pain.

The term “osteoarthritis” refers to a type of arthritis caused by inflammation, breakdown, and eventual loss of cartilage in the joints.

The term “therapeutically effective amount” is defined as the amount of a compound or pharmaceutical composition that will elicit a biological or medical response of a tissue, system, animal or human that is being sought by the researcher, medical doctor or other clinician.

The terms “treat,” “treating,” or “treatment” as used herein, refer to both therapeutic treatment and prophylactic or preventative measures, wherein the object is to prevent or slow down (i.e., lessen) an undesired physiological change or disease/disorder. Beneficial or desired clinical results include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

The term “pharmaceutically acceptable” when used in reference to a carrier, whether diluent or excipient, refers to a carrier that is compatible with the other ingredients of the formulation and not deleterious to the recipient thereof.

The terms “administration of a composition” or “administering a composition” are defined to include an act of providing a compound of the invention or pharmaceutical composition to the subject in need of treatment.

### B. Embodiments of the Invention

According to exemplary embodiments of the present invention, there are disclosed pharmaceutical compositions comprising a therapeutically effective quantity of a heparinoid, or a derivative or analog thereof, intended for use in treating, preventing or alleviating a disease, condition, syndrome, symptom, pathology, or malady in a subject in need thereof. In various embodiments, pharmaceutical compositions of the present application may be used to treat a disease or condition, including symptoms thereof, such as interstitial cystitis or osteoarthritis, as well as transmissible spongiform encephalopathy, immunodeficiency virus, hematomes, hemorrhoids, frostbites, burns, and multiparameter illnesses such as thrombosis and atherosclerosis.

i. Heparinoid

Pharmaceutical compositions disclosed herein comprise a therapeutically effective amount of a heparinoid. According to further embodiments, the heparinoid (as defined above) may be any of pentosan, heparin (the structures of both are shown above), hyaluronic acid, chondroitin, pharmaceutically acceptable salts thereof, and any combination thereof.

Those having ordinary skill in the art may also use (an)other heparinoid instead of, or in combination with, the above-named compound(s), if desired. The concentration of the heparinoid(s) described above, in the compositions, may be between about 0.5% mass % and 10.0 mass % of the total units of the suspension (weight/volume), for example, 1.0 mass %, 2.0 mass %, 3.0 mass %, 4.0 mass %, etc., up to and including about 10.0 mass %.

ii. Anhydrous Suspensions

According to further embodiments of the invention, the pharmaceutical compositions disclosed herein are formulated as stable, anhydrous suspensions that include a dispersed phase comprising a therapeutically effective quantity of a pharmaceutically acceptable heparinoid, and an anhydrous dispersion medium (which may optionally include at least one pharmaceutically acceptable surfactant, solubilizing agent, and/or suspending agent), in which the dispersed phase is dispersed.

As disclosed herein, the compositions according to these embodiments are in the form of a stable suspension. The suspensions include, consist of, or consist essentially of, an anhydrous dispersion medium (i.e., the continuous phase) and a dispersed phase that is dispersed within the dispersion medium. The dispersed phase includes, consists of, or consists essentially of, particles of a therapeutically effective quantity of at least one pharmaceutically acceptable heparinoid, or derivatives or analogs thereof. The dispersion medium includes at least one of a vegetable oil or a medium chain triglyceride. Suspensions of the present application are stable, meaning the concentration of heparinoid, as measured in a sample obtained from the suspension, does not deviate by more than about ±15%, e.g., ±10%, after storage at room temperature for at least 30, 60, 90, or 180 days. The concentration of heparinoid may be measured by routine analytical methods, including the method detailed in the examples, below.

In various embodiments, the suspension may also include one or more additional dispersed phases (e.g., a second dispersed phase, a third dispersed phase, etc.). For example, a first dispersed phase may have a first active ingredient and a second dispersed phase may have a second active ingredient, wherein at least one of the active ingredients is a heparinoid. As another example, both a first and second solid phase may have the same active ingredient (i.e., a heparinoid), but the particles may be formulated to provide different rates of drug release.

As mentioned above, the anhydrous dispersion medium of the composition of the present invention is comprised of at least vegetable oil or at least one medium chain triglyceride or a combination of products from both classes. The dispersion medium forms the major portion of the composition, its mass concentration in the composition being between about 90.0 mass and about 99.5 mass %, for example, about 99.0 mass %, 98.0 mass %, 97.0 mass %, or 96.0 mass %.

Specific examples of acceptable vegetable oils that may be used in the anhydrous dispersion medium according to various embodiments of the invention include, without limitation, castor oil, soybean oil, coconut oil, avocado oil, olive oil, almond oil, and combinations thereof. Those having ordinary skill in the art may use (an)other vegetable oil(s) instead of, or in combination with, those mentioned above, if desired. Those having ordinary skill in the art will understand that all these oils represent complex blends of organic compounds, as opposed to individual organic molecules.

For example, castor oil is a complex mixture of several fatty acids, principally, ricinoleic acid, an unsaturated, 18-carbon fatty acid having a hydroxyl functional group on the 12^{th} carbon (IUPAC, 12-hydroxyoctadec-9-enoic acid). Those having ordinary skill in the art will understand that castor oil has a very complex chemical structure and is a mixture of triglycerides that varies, but commonly comprises ricinoleic acid (about 70%) plus triglycerides of linoleic (IUPAC, 9,12-octadecadienoic) and oleic (IUPAC, octadec-9-enoic) acids (about 20% combined).

Almond oil is another complex mixture of several fatty acids, which varies, but typically comprises 65 to 70 mass % of oleic, 20 to 25% of linoleic, up to 4% of palmitic (IUPAC, hexadecanoic) and small quantities of palmitoleic (IUPAC, hexadec-9-enoic) and stearic (IUPAC, octadecanoic) acids.

Coconut oil is yet another complex mixture of several fatty acids, which also varies, but commonly comprises about 45 to 50% of lauric acid, the balance being a combination of other medium-chain fatty acids described above, as well as palmitic and stearic acids.

Olive oil is yet another mixture of several fatty acids, which also varies, but its principal ingredient is oleic acid (about 75 to 85%), the balance being a combination of other fatty acids including linoleic and palmitic acids.

A variety of medium-chain triglycerides can be used for forming the anhydrous dispersion medium. For example, triglyceride(s) containing the aliphatic tails derived from caprylic acid or caproic acid may be so used. Those having ordinary skill in the art may use (an)other medium-chain triglyceride(s) instead of, or in combination with, those based on caprylic or caproic acids, if desired. One specific product comprising medium-chain triglycerides that may be used is UNISPEND® anhydrous sweetened liquid (Fagron, Inc., St. Paul, Minn.). As stated above, the anhydrous dispersion medium used herein further optionally comprises at least one emulsifier or solubilizing and suspending agent which may be present in the compositions of the instant invention, if used, at mass concentrations between about 0.1 mass % and about 10.0 mass %, such as between about 1.0 mass % and about 5.0 mass %, for example, about 1.0 mass %, 2.0 mass %, 3.0 mass % or 4.0 mass %.

Those having ordinary skill in the art will choose the most appropriate emulsifier, solubilizing agent, and/or suspending agent, if one is to be used. For example, one such emulsifier, solubilizing agent, and/or suspending agent is a non-ionic polyoxyethlene-polyoxypropylene block copolymer having the general structure HO—(CH₂—CH₂—O)ₓ—(C₃H₆—O)_{y}—(CH₂—CH₂—O)ₓ—H,

wherein x is an integer having the value of at least 8 and y is an integer having the value of at least 38. Polyoxyethlene-polyoxypropylene block copolymer(s) that can be used may be those belonging to the PLURONIC® or POLOXAMER® families, chemically, poly(ethylene glycol)-block-poly(propylene glycol)-block-poly(ethylene glycol), both available from BASF Corp. and from several other vendors and having the following general chemical structure:

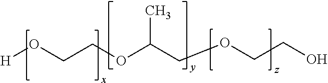

A specific and non-limiting example of a non-ionic polyoxyethlene-polyoxypropylene block copolymer that can be used is the product known under the trade name PLURONIC® which is described by the chemical structure above, with the molecular weight of the polyoxypropylene portion of about 1,750 Daltons, about a 40% polyoxyethylene content (mass), and the average overall molecular weight of about 2,900 Daltons. Another specific non-limiting example of a non-ionic polyoxyethlene-polyoxypropylene block copolymer that can be used is the product known under the trade name POLOXAMER 407® (also known as PLURONIC® F127), which is also described by the chemical structure above, with the molecular weight of the polyoxypropylene portion of about 4,000 Daltons, about a 70% polyoxyethylene content (mass), the overall molecular weight of between about 9,840 Daltons and about 14,600 Daltons.

Some non-limiting examples of other emulsifiers, solubilizing agents, and suspending agents that may be used in combination with, or instead of, non-ionic polyoxyethlene-polyoxypropylene block copolymers, include derivatives of cellulose, optionally partially cross-linked polyacrylates, polyoxyethylene sorbitan monolaurates, glyceryl isostearate, polyoxyethylene sorbitan monopalmitates, polyoxyethylene sorbitan monostearates, polyoxyethylene sorbitan monooleates (e.g., members of POLYSORBATE® family of products), glyceryl distearate, triglycerol monooleate, and polysaccharide thickening agents such as xanthan gum.

For example, suitable derivatives of cellulose that may be used include, without limitations, carboxymethyl cellulose, methyl cellulose, hydroxyethyl cellulose, and hydroxypropyl cellulose (Dow Chemical, Midland, Mich.). Examples of acceptable, partially cross-linked polyacrylates that may be used include, without limitations, polymers of the CARBOPOL® family (Lubrizol, Wickliffe, Ohio). Typically, the cross-linking agents that may be used to cross-link such polyacrylates are allyl sucrose or allyl pentaerythritol.

Suitable products of POLYSORBATE® family (i.e., ethoxylated sorbitan esterified with fatty acids) that may be used include, without limitations, polyoxyethylene sorbitan monolaurates, polyoxyethylene sorbitan monopalmitates, polyoxyethylene sorbitan monostearates, or polyoxyethylene sorbitan monooleates, some of which are also known as TWEEN® products, such as POLYSORBATE® 80) (Croda, Wilmington, Del.).

One typical product of the latter family that can be used is POLYSORBATE 80® (chemically, polyoxyethylene (20) sorbitan monooleate, also known as sorbitan mono-9-octadecenoate poly(oxy-1,2-ethanediyl), i.e., a product of polycondensation of polyethoxylated sorbitan and oleic acid having 20 units derived from ethylene glycol), which is a nonionic surfactant and emulsifier having the structure.

According to some embodiments, those having ordinary skill in the art will find it helpful to use the above-described anhydrous dispersions that are slightly acidic, such as those having a pH in the range of between about 5.0 and about 6.0. In additional embodiments, it may prove beneficial to use the above-described anhydrous dispersions that are homogenous, e.g., having the polydispersity index of about 2.0 or less. Methods of adjusting the pH of the suspensions as well as methods of purifying them to achieve the above-mentioned degree of polydispersity are well-known to those having ordinary skill in the art.

In some embodiments, the compositions disclosed herein may be optionally compounded as delayed or targeted release compositions. To that end, the compositions may further optionally include one or several pharmaceutically acceptable excipient(s) allowing delayed or controlled or targeted release of some or all of the heparinoid. For example, an excipient that can be used may be a polyacrylate dispersion, e.g., EUDRAGIT NE 30 D® (available from Evonik Industries, Parsippany, N.J.), which can be used for preparing the formulations in the form of a suspension to protect from gastric acid and delay pH dependent dissolution. Those skilled in the art may select another excipient for delayed or controlled release, if desired. The concentration of such excipient(s), if used, in the compositions may between about 50.0 mass % and about 80.0 mass % of the total mass of the suspension.

Another type of product that can be used in the excipient portion of the pharmaceutical formulation for preparing delayed release compositions may be water-soluble methylcellulose and hydroxypropyl methylcellulose polymers, such as METHOCEL® family of products, for example, a hydroxypropyl methylcellulose product METHOCEL® E4M.

In some additional embodiments, the effect of the delayed, controlled, and/or targeted release may be achieved by mixing the anhydrous suspension containing active components with the above-mentioned polyacrylate-based (e.g., EUDRAGIT®) or methylcellulose-based excipient (e.g., METHOCEL®). Alternatively, the anhydrous suspension maybe ensconced within the capsules made of such excipients.

Excipients that can be used for fabricating the pharmaceutical compositions described herein may also optionally include one or more of various taste modifiers such as sweeteners (e.g., sucrose and derivatives, sodium saccharin, aspartame, stevioside, monosodium glycyrrhizinate), flavoring agents (e.g., those introducing any natural or artificial fruit, vegetable, flower, beverage or candy flavor such as caramel, cherry, citrus (lemon, orange, tangerine), raspberry, vanillin and vanilla, marshmallow, chocolate, etc.), or anesthetic agents (e.g. menthol, peppermint, cinnamon).

Those having ordinary skill in the art will realize that yet (an)other additional emulsifier(s), solubilizing agent(s), and/or suspending agent(s) may be used, if desired, and will select such supplemental emulsifier(s), solubilizing agent(s), and/or suspending agent(s), as well as to choose the quantity thereof.

According to various embodiments of the present application, the pharmaceutical compositions described herein may be formulated as stable two-phase suspensions as defined above. More specifically, according to these embodiments, the suspensions consist of two phases, i.e., the dispersed phase that is dispersed within the dispersion medium. The dispersed phase includes particles comprising a therapeutically effective quantity of the pharmaceutically active component, i.e., a heparinoid described above.

In various embodiments, no compounds other than heparinoids described hereinabove are present within the particles that form the dispersed phase. According to such embodiments, the dispersion medium is a liquid that includes all other compounds that are present in the pharmaceutical compositions described in the application. In still further embodiments, the dispersion medium may be free of heparinoids, except for trace quantities.

In various embodiments, in addition to heparinoids, the dispersed phase may optionally contain other compounds, such as, without limitation, stabilizers, anti-oxidants, preservatives, various flavoring agents or sweeteners.

iii. Aqueous Solutions

As mentioned above, according to various embodiments of the present application, the pharmaceutical compositions described herein may be formulated as aqueous solutions. Such compositions comprise solutions of at least one heparinoid in water, with various water-soluble excipients and additives being optionally added to the solutions. Typically, such solutions contain between about 5.0 mass % and about 20.0 mass % of heparinoid(s). Those having ordinary skill in the art will determine which excipients or additives to add, if desired.

According to further embodiments, methods for fabricating the above-described pharmaceutical compositions are provided. A one-batch formulation method may be used, where the components of the pharmaceutical formulation can be combined in single container; the components may be added to the container simultaneously or consecutively. Those having ordinary skill in the art can choose the best method for preparing the compositions.

iv. Methods of Use

The compositions/medications prepared as described above may then be prescribed and given to a patient for oral or intravenous administration for treating, mitigating or preventing various diseases, conditions, syndromes, symptoms, pathologies, or maladies in a mammalian subject in need of such treatment, for example, for treating, mitigating or preventing interstitial cystitis or osteoarthritis.

Pharmaceutical formulations which are anhydrous dispersions, as described above, can typically be administered orally for treating, mitigating or preventing interstitial cystitis. In contrast, pharmaceutical formulations which are aqueous solutions, as described above, can typically be administered by intravenous injections for treating, mitigating or preventing osteoarthritis. An ordinarily skilled physician may prescribe delivery by any other acceptable method if so desired and indicated.

It will be understood by those having ordinary skill in the art that the specific dose level and frequency of dosage for any particular patient may be varied and will depend upon a variety of factors including the activity of the specific compound employed, the metabolic stability and length of action of that compound, the age, body weight, general health, diet, and the severity of the particular disease or condition being treated.

### C. Examples

### Example 1. Preparing a Pharmaceutical Composition No. 1

A pharmaceutical composition was prepared as described below. The following products were used in the amounts specified:

(a) about 1.0 g of solid powdered pentosan polysulfate sodium;

(b) about 1.5 mL of caramel liquid artificial flavor; and

(c) about 100 mL of UNISPEND® anhydrous sweetened medium chain triglyceride.

Pentosan polysulfate sodium was triturated using mortar and pestle according to standard technique of mixing solids. The artificial caramel flavor liquid was then added, with trituration followed by adding the anhydrous sweetened medium chain triglyceride (i.e., UNISPEND®), and by transferring the product to the dispensing bottle.

Finally, the mortar was washed using a small quantity of anhydrous sweetened medium chain triglyceride, and the wash was transferred to a bottle, to ensure the entire quantity of active components has been so transferred followed by packaging and labeling.

The composition was then tested chromatographically for stability after the storage for 0 to 180 days at room temperature. At least 90% of the original pentosan polysulfate sodium was still preserved intact after 180 days, as shown by the data in Table 1 below.

TABLE 1
Stability Data for the Anhydrous Dispersion of Pentosan
Polysulfate Sodium
Quantity of Pentosan Polysulfate Sodium, % of the
Original Quantity, After Storage at Room Temperature

| Day 0 | Day 30 | Day 60 | Day 90 | Day 120 | Day 150 | Day 180 |
|---|---|---|---|---|---|---|
| 95 | 107 | 97 | 87 | 94 | 95 | 90 |

### Example 2. Preparing a Pharmaceutical Composition No. 2

A pharmaceutical composition may be prepared as described below. The following products may be used in the amounts specified:

(a) about 10.0 g of solid powdered pentosan polysulfate sodium;

(b) about 1.0 mL of benzyl alcohol;

(c) about 0.68 g of dibasic anhydrous sodium phosphate;

(d) about 0.22 g of monobasic anhydrous sodium phosphate;

(e) about 100.0 mL of water sterile for injection; and

(f) a small quantity of sodium hydroxide solution (for adjusting pH).

Pentosan polysulfate sodium may be dissolved in about 85% of water, followed by adding and dissolving with continued mixing, one by one, dibasic anhydrous sodium phosphate, monobasic anhydrous sodium phosphate, and benzyl alcohol. If necessary, the pH of the solution may be then adjusted to 5.3-6.0 by adding the sodium hydroxide solution in the dropwise manner. The remainder of water then can be added to obtain the final product, followed by transferring the solution into the sterile serum bottle using a 0.22 micron filter, packaging and labeling.

### Example 3. Preparing a Pharmaceutical Composition No. 3 (Delayed Release Suspension)

A pharmaceutical composition may be prepared as described below. The following products may be used in the quantities specified:

(a) about 2% (mass) of solid powdered pentosan polysulfate sodium;

(b) about 6.25% (mass) of EUDRAGIT L polymer;

(c) about 1.5% (mass) of caramel liquid artificial flavor; and

(d) the balance, UNISPEND® anhydrous sweetened medium chain triglyceride.

The above-mentioned dry ingredients may be combined in 90% of final volume of anhydrous vehicle, followed by homogenization for 10 minutes and adding the remainder of the anhydrous vehicle, quantum sufficit to 100.0 g.

### Example 4. Preparing a Pharmaceutical Composition No. 4 (Capsules)

A pharmaceutical composition may be prepared as described below. The following products may be used in the quantities specified:

(a) about 0.2 g of solid powdered pentosan polysulfate sodium;

(b) about 0.2 g of METHOCEL® E4M polymer;

(c) about 0.05 g METHOCEL® K100 polymer; and

(d) microcrystalline cellulose, quantum sufficit to 0.5 g.

Powdered solid pentosan polysulfate sodium may be ground to a fine powdery consistency using a mortar and pestle, and then, using the principles of geometric dilution, which are known to those having ordinary skill in the art, mixed with the ingredients (b), (c), and (d) together with trituration in a mortar. A V-blender and powder food coloring is to be used to verify homogenous mixture of the powders.

Capsule formulations should have powders where the particle size is the same throughout. Once powders are thoroughly mixed, the mixture can be sieved through an 80 mesh sieve to ensure even particle size. Large particles are not to be forced through the sieve as this destroys the integrity of the sieve. Instead, any particles remaining in the sieve are to be triturated in a mortar and pestle to reduce the particle size, and the mixture of all the powders should be sieved again. The final product can then be encapsulated in size #1 capsules.

Although the invention has been described with reference to the above examples, it will be understood that modifications and variations are encompassed within the spirit and scope of the invention. Accordingly, the invention is limited only by the following claims.

What is claimed is:

1. A pharmaceutical composition formulated as an anhydrous suspension comprising:
(a) a dispersed phase comprising a therapeutically effective quantity of a pharmaceutically acceptable heparinoid; and
(b) an anhydrous dispersion medium optionally comprising at least one pharmaceutically acceptable surfactant, solubilizing agent, and/or suspending agent,
wherein the dispersed phase is dispersed within the dispersion medium.

2. The pharmaceutical composition of claim 1, wherein the heparinoid is selected from the group consisting of pentosan, heparin, hyaluronic acid, chondroitin, pharmaceutically acceptable salts thereof, and any combination thereof.

3. The pharmaceutical composition of claim 2, wherein the heparinoid is pentosan polysulfate sodium.

4. The pharmaceutical composition of claim 3, wherein the concentration of pentosan polysulfate sodium in the composition is between about 0.5 mass % and about 10.0 mass %.

5. The pharmaceutical composition of claim 1, wherein the anhydrous dispersion medium comprises at least one vegetable oil or at least one medium chain triglyceride, or any combination thereof.

6. The pharmaceutical composition of claim 5, wherein the vegetable oil is selected from the group consisting of castor oil, soybean oil, coconut oil, avocado oil, olive oil, almond oil, and combinations thereof.

7. The pharmaceutical composition of claim 5, wherein the medium chain triglyceride is a triglyceride having at least two of the three fatty acid moieties that are derived from saturated open-chain acids having between 6 and 12 carbon atoms.

8. The pharmaceutical composition of claim 7, wherein the saturated open-chain acids are selected from the group consisting of caprylic acid and caproic acid.

9. The pharmaceutical composition of claim 1, wherein the surfactant, solubilizing agent, and/or suspending agent is selected from the group consisting of non-ionic polyoxyethlene-polyoxypropylene block copolymers, a water-soluble derivative of cellulose, optionally partially cross-linked polyacrylates, polyoxyethylene sorbitan monolaurates, glyceryl distearate, triglycerol monooleate, glyceryl isostearate, polyoxyethylene sorbitan monopalmitates, polyoxyethylene sorbitan monostearates, and polyoxyethylene sorbitan monooleates.

10. The pharmaceutical composition of claim 9, wherein the non-ionic polyoxyethlene-polyoxypropylene block copolymer is poly(ethylene glycol)-block-poly(propylene glycol)-block-poly(ethylene glycol).

11. The pharmaceutical composition of claim 9, wherein the water-soluble derivative of cellulose is selected from the group consisting of carboxymethyl cellulose, methyl cellulose, hydroxyethyl cellulose, and hydroxypropyl cellulose.

12. The pharmaceutical composition of claim 9, wherein the solubilizing agent and/or suspending agent is polyoxyethylene (20) sorbitan monooleate.

13. The pharmaceutical composition of claim 1, further comprising at least one taste modifier selected from the group consisting of sweeteners, flavoring agents, and anesthetic agents.

14. A pharmaceutical composition comprising a therapeutically effective quantity of a pharmaceutically acceptable heparinoid and an excipient that enables delayed release or sustained release of the heparinoid.

15. The pharmaceutical composition of claim 14, wherein the excipient comprises a polyacrylate dispersion or at least one water-soluble polymer selected from the group consisting of methylcellulose and hydroxypropyl methylcellulose, wherein the composition is formulated as a delayed release suspension or as an encapsulated solid product.

16. A method for treating, preventing or alleviating interstitial cystitis in a patient in need of such treatment comprising orally administering to the subject the composition of claim 1.

17. A method for treating, preventing or alleviating osteoarthritis comprising administering to the patient by an injection an aqueous composition comprising a therapeutically effective quantity of a pharmaceutically acceptable heparinoid, wherein the injection is selected from the group consisting of an intravenous injection, intramuscular injection and intraarticular injection.

18. The pharmaceutical composition of claim 17, wherein the heparinoid is selected from the group consisting of pentosan, heparin, hyaluronic acid, chondroitin, pharmaceutically acceptable salts thereof, and any combination thereof.

19. The pharmaceutical composition of claim 18, wherein the heparinoid is pentosan polysulfate sodium.

20. The pharmaceutical composition of claim 19, wherein the concentration of pentosan polysulfate sodium in the composition is between about 5.0 mass % and about 20.0 mass %.

21. A pharmaceutical composition formulated as an anhydrous suspension comprising:
(a) a dispersed phase comprising a therapeutically effective quantity of a pharmaceutically acceptable heparinoid; and
(b) an anhydrous dispersion medium;
wherein the concentration of heparinoid in the suspension does not deviate by more than ±10%, after storage at room temperature for at least 30 days.

22. The pharmaceutical composition of claim 21, wherein the concentration of heparinoid in the suspension does not deviate by more than ±10%, after storage at room temperature for at least 60 days.

23. The pharmaceutical composition of claim 21, wherein the concentration of heparinoid in the suspension does not deviate by more than ±10%, after storage at room temperature for at least 90 days.

24. The pharmaceutical composition of claim 21, wherein the concentration of heparinoid in the suspension does not deviate by more than ±10%, after storage at room temperature for at least 180 days.

25. The pharmaceutical composition of claim 21, wherein the heparinoid is selected from the group consisting of pentosan, heparin, hyaluronic acid, chondroitin, pharmaceutically acceptable salts thereof, and any combination thereof.

26. The pharmaceutical composition of claim 25, wherein the heparinoid is pentosan polysulfate sodium.

27. The pharmaceutical composition of claim 26, wherein the concentration of pentosan polysulfate sodium in the composition is between about 0.5 mass % and about 10.0 mass %.

28. The pharmaceutical composition of claim 21, wherein the anhydrous dispersion medium comprises at least one vegetable oil or at least one medium chain triglyceride, or any combination thereof.

29. The pharmaceutical composition of claim 1, wherein the heparinoid has the polydispersity index of 2.0 or less.
